(19) United States
(12) Patent Application Publication
(10) Pub. No.: US 2004/0127476 A1
(43) Pub. Date: Jul. 1, 2004

(54) Oral testosterone delivery system with improved sustained release

(76) Inventors: Alvin Kershman, Paradise Valley, MO (US); Jeff L. Shear, Chesterfield, MO (US)

Correspondence Address: Greensfelder, Hemker & Gale, P.C., Intellectual Property Practice Group, Suite 2000, 10 South Broadway, St. Louis, MO, 63102, US

(21) Appl. No.: 10/706,241

(22) Filed: Nov. 12, 2003

Related U.S. Application Data
(60) Provisional application No. 60/426,188, filed on Nov. 14, 2002.

(30) Foreign Application Priority Data
Nov. 12, 2003 (WO) PCT/US03/35899

(51) Int. Cl. A61K 31/57; A61K 9/26; A61K 9/16

(52) U.S. Cl. 514/177; 424/469

(57) ABSTRACT

An oral testosterone delivery system with sustained release properties where the micronized testosterone is present as a tablet, bolus, capsule, powder, liquid or suspension and, optionally, at least part of the testosterone is microencapsulated; in a preferred embodiment, the testosterone is present in a solid lipid suspension, wherein the suspension exhibits pseudotropic and/or thixotropic flow properties when melted.

### CROSS-REFERENCE TO RELATED APPLICATION

This application is based upon and claims the priority of provisional application Serial No. 60/426,188, filed 14 Nov. 2002, entitled Oral Testosterone Delivery System with Improved Sustained Release of the present inventors, which is incorporated by reference. Continued preservation of said provisional application is requested.

### FIELD OF THE INVENTION

The present invention relates to an oral testosterone delivery system that provides improved sustained release of testosterone. Said delivery system includes both human and veterinary applications.

### BACKGROUND OF THE INVENTION

Drug efficacy generally depends upon the ability of the drug to reach its target in sufficient quantity to maintain therapeutic levels for the desired time period. Orally administered drugs must overcome several obstacles to reach their desired targets. Before orally administered drugs enter the general circulation of the human body, they are absorbed into the capillaries and veins of the upper gastrointestinal tract and are transported by the portal vein to the liver. The pH and enzymatic activities found in gastrointestinal fluids may inactivate the drug or cause the drug to dissolve poorly and not be absorbed. In addition, following their absorption in the intestine, orally administered drugs are often subject to a “first pass” clearance by the liver and excreted into bile or converted into pharmacologically inactive metabolites.

The oral administration of hormones, such as testosterone or estrogen, have proven challenging. Testosterone is administered orally in a bonded form as testosterone undecanoate, methyltestosterone, or testosterone cyclodextrin, to avoid the first pass effect. When administered in a regiment of hormone replacement therapy, it is desired to have sustained release properties, yet these forms of testosterone must be taken multiple times daily.

Of particular interest is the delivery of the native form of testosterone. The native form of testosterone is more stable than its bonded predecessors. More of the active ingredient is delivered in a smaller dosage and tablet form. It is a simpler and less expensive manufacturing process that eliminates the additional step of bonding the testosterone. Further, the present dosage may be administered with or without food, unlike the bonded form which typically is administered with food consumption.

It is generally believed that testosterone cannot be administered orally. According to The Pharmacological Basis of Therapeutics, 10^{th} ed., by Goodman and Gilman, oral administration of testosterone leads to absorption into the hepatic circulation but results in rapid catabolism by the liver. Therefore, oral ingestion is ineffective in delivering testosterone systemically. However, some researchers have found conflicting evidence otherwise.

Svend Johnsen et al., in the publication entitled “Therapeutic Effectiveness of Oral Testosterone,” disclose the oral administration of 200 mg of micronized testosterone, with a particle size in the range of 2 to 5 microns, to four patients with no testicular function. It Was found that, for a period of about 5 to 7 hours, the total serum testosterone of the patient was in the range of about 300 to 900 ng/dL. Johnsen et al. recommended 200 mg testosterone administered twice daily. Johnsen et al. failed to address improving the sustained release properties of testosterone in order to administer the dose only once a day.

Marie Føgh et al., in the publication entitled “Serum-Testosterone During Oral Administration of Testosterone in Hypogonadal Men and Transsexual Women,” disclose the oral administration of 200 mg of micronized testosterone twice daily. The two doses provided total serum testosterone within the normal range for greater than about 12 h. A single 200 mg dose of orally administered testosterone with a particle size in the range of about 125-400 microns provided a total serum testosterone in the normal range for from about 5 to 7 hours. In view of the large doses required to maintain the desired serum levels of testosterone, and the possible side effects of such doses, Føgh et al. recommended not administering testosterone orally.

P. R. Daggett et al., in the article entitled “Oral Testosterone, a Reappraisal,” disclose the oral administration of 200 mg of micronized testosterone twice daily. The dosage provided a double peak effect, with a desired level of serum testosterone for about 4 hours for each peak. Daggett et al. found that the administration of oral testosterone was “unsuitable for routine use.”

Nieschlag et al., in the publication entitled “Influence of Sex, Testicular Development and Liver Function on the Bioavailability of Oral Testosterone,” disclose orally administering 63 mg of testosterone in arachis oil to hypogonadal men. The serum level of testosterone rose to the desired level for a period of about 1 to 2 hours. Nieschlag et al. stated that oral testosterone “should be considered with caution, since higher testosterone doses would be needed to exceed the developing capacity of the liver to metabolize testosterone.”

In view of the fact that it is generally believed that testosterone cannot be orally administered, none of the above references even discussed the possibility of improving sustained release properties.

“Sustained Release” generally refers to release of a drug whereby the level of drug available to the patient is maintained at some level Over a desired period of time. A variety of methods and formulations are used to provide sustained release of drugs. Some of the methods are disclosed in U.S. Pat. No. 5,567,439, which is hereby incorporated by reference. The desired level of total serum testosterone is in the range of about 250 to 1100 ng/dL. The present invention delivers the desired level of serum testosterone for from about 6 to 12 hours or more. Additionally, the present invention provides an improvement in sustained release properties of micronized testosterone of about at least 10%.

None of the above-referenced patents describe the present invention of an orally administered testosterone with improved sustained release properties, as disclosed and claimed herein.

### SUMMARY OF THE INVENTION

The present invention comprises an oral delivery system for testosterone having improved sustained release properties. In the delivery system, the testosterone may be delivered as a tablet, capsule, bolus, liquid solution or suspension, or a mixture of dry ingredients. The testosterone, when delivered may provide total serum testosterone in the range of from about 250 to about 1100 ng/dL for a period of about six (6) to twelve (12) or more hours. The testosterone, when delivered in the present invention, may provide improved sustained release properties with an improvement of at least 10% over that shown in the art using micronized testosterone alone or in a gelatin capsule. Further improvements in delivery may provide total serum testosterone delivered in the range of about 250 to 1100 ng/dL for a period of about eight (8) to about (15) hours or more.

The present invention contemplates any sustained release system that is known to increase the release time of testosterone such that the total serum testosterone falls in the desired range. Such systems include filling a polymer capsule with a solid, liquid, suspension or gel that contains testosterone which may be slowly released by diffusion through the capsule walls. Heterogeneous matrices, for example compressed tablets, may control the release of testosterone either by diffusion or erosion of the matrix of a combination. Laminates of polymeric material and testosterone may be formed into a sandwich, and diffusion or erosion controls the release of testosterone. Liquid-Liquid encapsulated in a viscous syrup-like solution of polymer may control the release of testosterone. Heterogeneous dispersions of testosterone in water-swellable hydrogen matrices may control the release of testosterone by slow surface-to-center swelling of the matrix and subsequent diffusion of the agent from the water-swollen part of the matrix. Other systems may use waxes or lipids to prolong the release of the testosterone. A preferred delivery system uses a lipid suspension to deliver the testosterone.

### DETAILED DESCRIPTION OF THE INVENTION

The Lipid Suspension

One embodiment of the invention is a solid lipid suspension. The lipids of the present invention may be of animal, vegetable or mineral origin, which are substantially water-insoluble, inert, non-toxic hydrocarbon fats and oils and derivatives thereof, and may comprise any of the commonly commercially available fats or oils approved by the Food & Drug Administration, having melting points in the range of about 90 to 160° F. (32 to 71° C.). The lipid may comprise a vegetable oil base commonly known as hard butter. Hard butters are hydrogenated, press fractionated, or other processed oils that are processed or recombined to have a solid fat index (percent solid fat vs. temperature) similar to that of cocoa butter. However, other lipids may be used that are relatively hard or solid at room temperature, but melt rapidly in the mouth at a temperature of about 92°to 98° F. (29 to 32° C.)(mouth temperature). The lipid is employed in the amounts within the range of from about 20 to 50%. Above about 50%, the suspension flows too readily and does not exhibit thixotropic or pseudoplastic flow properties. When present below about 20%, the amount of lipid is not sufficient to completely coat the dry particles.

Examples of suitable lipids include tallow, hydrogenated tallow, hydrogenated vegetable oil, almond oil, coconut oil, corn oil, cottonseed oil, light liquid petrolatum, heavy liquid petrolatum, olein, olive oil, palm oil, peanut oil, persic oil, sesame oil, soybean oil or safflower oil. Additionally, stearines can be used as a lipid in the present invention. The addition of stearines to the product provides the favorable property of mold-release. Further, the addition of stearines raises the melting point of the composition as high as about 100° F. (38° C.), which is particularly beneficial when the product is shipped or stored in unrefrigerated compartments.

The fillers of the present invention are pharmacologically inert and optionally nutritionally beneficial to humans and animals. Such fillers include cellulose such as microcrystalline cellulose, grain starches such as cornstarch, tapioca, dextrin, sugars and sugar alcohols such as sucrose sorbitol, xylitol, mannitol and the like. Preferred fillers include non-fat milk powder, whey, grain brans such as oat bran, and fruit and vegetable pulps. Preferred fillers are finely divided and have a preferred average particle size in the range of about 0.10 to 500 microns. The fillers are present in the drug delivery device in a concentration of about 50 to 80%. Optionally, the pharmaceutical particles can also serve as filler in the delivery system.

Optionally, an emulsifier or surfactant may be used in the lipid suspension. Any emulsifier or surfactant approved for use in foods by the Food and Drug Administration and having a relatively low HLB value, in the range of about 1 to 3, is suitable for use in the present invention. The appropriate surfactant minimizes the surface tension of the lipid, allowing it to oil wet and encapsulate the non-oil solid particles. Typically, the surfactant is present in the delivery system in the concentration of about 0.1 to 1.0%. Suitable surfactants include alkyl aryl sulfonate, alkyl sulfonates, sulfonated amides or amines, sulfated or sulfonated esters or ethers, alkyl sulfonates, of dioctyl sulfonosuccinate and the like, a hydrated aluminum silicate such as bentonite or kaolin, triglycerol monostearate, triglycerol monoshortening, monodiglyceride propylene glycol, octaglycerol monooleate, octaglyceron monostearate, and decaglycerol decaoleate. The preferred surfactant is lecithin.

In a preferred embodiment, the testosterone is microencapsulated. Such microencapsulation includes sustained release encapsulation. Any known method of encapsulation is suitable in the present invention. Such methods include, but are not limited to air coating, chemical erosion, coacervation, fluid bed coating, macroencapsulation, microencapsulation, osmosis, pan spray coating, physical erosion, polymer protein conjugate systems, and polymeric microspheres. A preferred method involves slowly blending the drug with a filming agent solution to form granulated particles. The granulated particles are allowed to dry on a tray and are sieved to the desired size, typically in the range of from about 200 to 500 microns. The coating materials include, but are not limited to, acrylic polymers and co-polymers, alginates, calcium stearate, cellulose, including methylcellulose, ethylcellulose, and hydroxypropyl cellulose, gelatins, glyceryl behenate, glycholic acid and its various forms, ion exchange resins, lactic acid and its various forms, lipids, methacrylic monomers, methacrylic polymers and co-polymers, polyethylene glycol polymers, shellac (pharmaceutical glaze), stearic acid, glycerol esters of fatty acids and waxes. It is contemplated in the present invention that the microencapsulated testosterone may be used alone, or in the lipid suspension. Further, the microencapsulated testosterone may be used in any other system, such as tablets, boluses, enclosed in a gelatin capsule, or in a liquid or syrup system.

In another embodiment of the present invention, the testosterone is not microencapsulated, but suspended in the lipid as dry particles. Typically the testosterone is present in the delivery device in a concentration of 30% or less. However, the testosterone can comprise all of the dried particles, to provide the necessary dose.

Optionally, the dry particles include flavorings that make the device taste and smell appealing to humans or animals. The flavorings can be natural or synthetic, and can include fruit flavorings, citrus, meat, chocolate, vanilla, fish, butter, milk, cream, egg or cheese. The flavorings are typically present in the device in the range of about 0.05 to 50.0%.

The delivery device may also include other pharmaceutically acceptable agents, such as sweetening agents, including hydrogenated starch hydrolysates, synthetic sweeteners such as sorbitol, xylitol, saccharin salts, L-aspartyl-L-phenylalanine methyl ester, as well as coloring agents, other binding agents, lubricants, such as calcium stearate, stearic acid, magnesium stearate, antioxidants such as butylated hydroxy toluene, antiflatuants such as simethicone and the like.

Optionally, rupturing agents are used to rapidly deliver the testosterone into the recipient's system. A typical rupturing agent is a starch that swells in the presence of water. Various modified starches, such as carboxymethyl starch, currently marketed under the trade name Explotab or Primojel are used as rupturing agents. A preferred rupturing agent is sodium starch glycolate. When ingested, the capsule or pellet swells in the presence of gastric juices and ruptures.

In one embodiment of the present invention, the rupturing agent is present inside the microcapsule. As water penetrates the microcapsule, it swells the starch and ruptures the capsule, rapidly delivering the testosterone to the system. Additional rupturing agents are disclosed in U.S. Pat. No. 5,567,439, which is hereby incorporated by reference.

In another embodiment, the rupturing agent is present in the lipid suspension, which ruptures the pellet, but leaves the microcapsules intact. This allows the delayed delivery of the drug farther along in the digestive system, or in the intestines. The present invention is particularly effective in this embodiment, in that the ingested pellet may be chewable, where the pellet cleaves in the lipid suspension when chewed, but leaves the microcapsules intact. Tablets or gel capsules, when chewed, typically result in damage to or rupturing of the microcapsules defeating the effectiveness of the microcapsules.

In yet another embodiment, multiple drugs have multiple encapsulations, each containing an rupturing agent. The filming agents used for encapsulation are selected to disintegrate at selected pH conditions, which rupture and release each drug at desired locations in the digestive system.

The process for preparing the above delivery system comprises melting the lipid and mixing with the surfactant. The dry particles are mixed with the melted lipid mixture to form a suspension exhibiting pseudoplastic and/or thixotropic flow properties, and poured or molded to provide solid dosage forms.

The dry particles, which include the testosterone, filler and optional flavorings and additives, are pre-blended and typically have a particle size in the range of from about 50 to 150 microns. The pre-blended particles are gradually added to the heated lipid base until a high solid suspension is obtained, typically in the range of about 50 to 80% particles and from about 50 to 20% lipid. The preferred form of testosterone is micronized testosterone.

Slow addition of the dry particles is critical in the production of the device, to insure that the particles are suspended in their micronized state and riot as agglomerated clumps. Moreover, rapid addition can cause the mixing process to fail in that the melted suspension will not have the desired flow properties, but instead will be a granular oily mass (a sign of product failure). The mixing step is accomplished in a heated mixing device that insures thorough mixing of all materials with minimal shear, such as a planetary mixer or a scrape surface mixer. After the suspension is formed, the product is poured into molds and allowed to cool. De-molding and packaging are then performed. Alternatively, the suspension can be super-cooled and sheeted in a semi-soft format. The sheet is processed through forming rolls containing a design or configuration that embosses and forms the final shape.

The present invention includes filling a polymer capsule with a solid, liquid, suspension or gel that contains testosterone which may be slowly released by diffusion through the capsule walls. Heterogeneous matrices, for example compressed tablets, may control the release of testosterone either by diffusion or erosion of the matrix of a combination. Laminates of polymeric material and testosterone may be formed into a sandwich, and diffusion or erosion controls the release of testosterone. Liquid-Liquid encapsulated in a viscous syrup-like solution of polymer may control the release of testosterone. Heterogeneous dispersions of testosterone in water-swellable hydrogen matrices may control the release of testosterone by slow surface-to-center swelling of the matrix and subsequent diffusion of the agent from the water-swollen part of the matrix. Other systems may use liquid lipids to prolong the release of the testosterone. Hydrophilic gums may be used as carriers to sustain the release of testosterone.

The following examples are to illustrate the claimed invention and are not intended to limit the claims in any way. All of the percentages are by weight unless otherwise indicated.

### EXAMPLES

Example I was prepared according to the following procedure.

Forming the Suspension

The lipid (hydrogenated vegetable oil sold under the trademark KLX®) was heated in a Hobart 5 Quart planetary mixer jacketed with a heating mantle in the range of about 140 to 150° F. (60 to 66° C.) and melted. The surfactant, lecithin, was added to the lipid with mixing, and the mixture was allowed to cool to about 135° F. (° C.).

The dry particles, including the pharmaceutical (micronized, i.e., 3 to 5 microns, testosterone), the rupturing agent (sodium starch glycolate, sold under the trademark Explotab), and fillers (microcrystalline cellulose, sold under the trademark Eudragit s100, dry milk, salt and powdered sugar) were screened to a particle size in the range of about 200 and 500 microns and dry-blended. The dry particles were slowly added incrementally to the lipid/surfactant mixture with mixing over a period of about 1 hour, to provide a smooth suspension with no lumps or agglomerations. The suspension exhibited thixotropic and pseudoplastic flow properties. It was molded and cooled to about 70° F. (21° C.). The suspension shrank as it cooled, and easily released from the mold when inverted.

TABLE 1
Forming a Suspension of
Testosterone in a 250 mg Dose
BATCH FORMULA

| Ingredient | Weight (grams) | % |
|---|---|---|
| KLX (lipid) | 36.100 | 38.00 |
| Explotab (rupturing agent) | 4.750 | 5.00 |
| Eudragit s100 (cellulose) | 4.750 | 5.00 |
| Dry milk, low heat (filler) | 9.500 | 10.00 |
| Powdered sugar (filler) | 14.250 | 15.00 |
| Lecithin (surfactant) | 0.950 | 1.00 |
| Salt | 0.190 | 0.20 |
| Testosterone | 24.938 | 26.25 |
| Totals | 95 | 100.45 |

### Example 1

### Varying the Testosterone Dose 25, 50, 100, 250 mg

In vivo Evaluation:

A study using six dogs (female beagles) was made to obtain preliminary pharmacokinetic data following a single oral dose of the delivery system. The dogs were 13-24 months old, and weighed in the range of 10.4 to 13.2 kg.

The dosing was done in four sequential one day intervals with a minimum two day rest period in between each interval. Blood was drawn immediately before the dose was administered. The results revealed minimal levels of testosterone. The animals were given the placebo or test article, as described above, at approximately the same time each day, immediately prior to being fed. The dog ate its food within 30 minutes of the dose being administered.

Blood samples were collected pre-dose and at 0.5, 1, 2, 4, 5, 6, 8 and 24 hours post dosing. At each time point, a minimum of 3 mL whole blood (or minimum volume determined by assay requirement) were collected by venipuncture of the jugular vein into non-heparinized Vacutainer tubes. The blood was centrifuged to obtain serum, which was kept on ice until placed into an appropriately sized vial, and frozen at −70° C. The samples remained frozen until delivered on dry ice to the lab for analysis. The lab used radioimmunoassay to analyze for testosterone.

Example 1 Results:

TABLE 2
Average Serum Testosterone (ng/dL)

| | Testosterone Dose (mg) | | | |
|---|---|---|---|---|
| | 25 | 50 | 100 | 250 |
| | Testosterone | Testosterone | Testosterone | Testosterone |
| Time (h) | (ng/dL) | (ng/dL) | (ng/dL) | (ng/dL) |
| 0 | 0 | 1 | 0 | 26 |
| 0.5 | 286 | 154 | 270 | 264 |
| 1 | 390 | 286 | 309 | 555 |
| 2 | 425 | 376 | 450 | 835 |
| 4 | 118 | 288 | 522 | 1032 |
| 5 | 35 | 215 | 618 | 829 |
| 6 | 53 | 107 | 357 | 980 |
| 8 | 23 | 54 | 422 | 757 |
| 24 | 1 | 7 | 2 | 8 |

### Control 1

### Varying the Testosterone Dose 25, 50, 100, 250 mg in a Gel Capsule

Micronized testosterone was placed in a gelatin capsule and administered to dogs as described in Example 1. The results are summarized in Table 3.

Control 1 Results:

TABLE 3
Average Serum Testosterone (ng/dL)

| | Testosterone Dose (mg) | | | |
|---|---|---|---|---|
| | 25 | 50 | 100 | 250 |
| | Testosterone | Testosterone | Testosterone | Testosterone |
| Time (h) | (ng/dL) | (ng/dL) | (ng/dL) | (ng/dL) |
| 0 | 49 | 0 | 0 | 7 |
| 0.5 | 253 | 150 | 772 | 1315 |
| 1 | 664 | 204 | 916 | 1306 |
| 2 | 238 | 324 | 703 | 1786 |
| 4 | 123 | 266 | 372 | 1009 |
| 5 | 109 | 293 | 332 | 775 |
| 6 | 57 | 295 | 278 | 542 |
| 8 | 20 | 165 | 143 | 412 |
| 24 | 1 | 3 | 2 | 16 |

A comparison of the sustained release properties of Example 1 and Control 1 is given in Table 4. The comparison is made by evaluating the amount of time the blood serum levels fell between about 250 and 1100 ng/dL.

TABLE 4
Sustained Release Times
Example 1 and Control 1

| Testosterone | Lipid Suspension | Gelatin Capsule |
|---|---|---|
| Dose (mg) | Time (h), Example 1 | Time (h), Control 1 |
| 25 | 1.5 | 0.5 |
| 50 | 3.0 | 4.0 |
| 100 | 7.5 | 5.5 |
| 250 | 7.5 | 4.0 |

A clear improvement is noted for doses of 100 mg and higher. Smaller doses fail to maintain the desired levels for a sufficient length of time. It is important to note that the present data is taken using dogs as test animals. It is generally recognized that the metabolism of dogs is higher than that of humans, and that humans will typically display higher blood serum levels for a greater period of time under similar test conditions. It is expected that humans will experience even greater sustained release levels than those shown in the dogs.

### Example 2

### Varying the Amount of Rupturing Agent

Samples of a lipid suspension were prepared as in Example 1, wherein the amount of testosterone administered was 250 mg, and the amount of rupturing agent was varied as follows: 0, 1, 2 and 5%.

In vivo Evaluation:

A study using four dogs (female beagles) was made to obtain preliminary pharmacokinetic data following a single oral dose of the delivery system. The dogs were over 18 months old, and weighed in the range of 11.1 to 12.6 kg.

The dosing was done in four sequential one day intervals with a minimum four day rest period in between each interval. Blood was drawn immediately before the dose was administered. The results revealed minimal levels of testosterone. The animals were given the placebo or test article, as described above, at approximately the same time each day, immediately prior to being fed. The dog ate its food within 30 minutes of the dose being administered.

Blood samples were collected pre-dose and at 3, 6, 8, 10, 12, 16, 20 and 24 hours post dosing. At each time point, a minimum of 3 mL whole blood (or minimum volume determined by assay requirement) were collected by venipuncture of the jugular vein into non-heparinized Vacutainer tubes. The blood was centrifuged to obtain serum, which was kept on ice until placed into an appropriately sized vial, and frozen at −70° C. The samples remained frozen until delivered on dry ice to the lab for analysis. The lab used radioimmunoassay to analyze for testosterone.

Test Results:

TABLE 5
Average Serum Testosterone (ng/dL)

% Explotab*

| Time (h) | 5 | 0 | 1 | 2 |
|---|---|---|---|---|
| 0 | 2.0 | 0.0 | 2.5 | 0.3 |
| 3 | 433.5 | 485.8 | 274.0 | 690.8 |
| 6 | 1257.0 | 537.3 | 561.3 | 920.0 |
| 8 | 479.8 | 520.8 | 772.5 | 776.0 |
| 10 | 330.3 | 410.5 | 553.3 | 840.0 |
| 12 | 224.5 | 243.5 | 449.3 | 293.8 |
| 16 | 31.5 | 213.0 | 212.8 | 61.3 |
| 20 | 12.0 | 72.3 | 88.0 | 29.0 |
| 24 | 6.8 | 48.3 | 54.5 | 27.3 |

*The rupturing agent.

Each dose, for a period of time, is above 250 ng/dL average serum testosterone. The samples in Example 2 demonstrate improved sustained release properties, maintaining the desired levels of serum testosterone from about 7 to 9 h. The sample with 5% Explotab had one serum level of testosterone exceeding 1100 ng/dL.

### Example 3

### Varying the Surfactant

An in vivo evaluation, of the present invention was made, using the formulation from Table 1, but varying the surfactant as follows. The same procedure was followed as described in Example 3, except that three dogs were used and there was a two day washout.

TABLE 6
Average Serum Testosterone (ng/dL)

Surfactant

| Time (h) | Lecithin | No Surfactant | Durem 300* |
|---|---|---|---|
| 0 | 1.1 | 0.0 | 0.0 |
| 0.5 | 51.3 | 94.3 | 104.7 |
| 1.0 | 397.7 | 277.3 | 217.7 |
| 2.0 | 929.3 | 609.7 | 1136.7 |
| 4.0 | 1558.0 | 1410.0 | 581.0 |
| 5.0 | 1561.3 | 702.3 | 591.0 |
| 6.0 | 1039.3 | 632.7 | 688.7 |
| 8.0 | 502.0 | 375.0 | 576.3 |
| 24.0 | 10.0 | 48.0 | 59.3 |

*Monodiglyceride propylene glycol surfactant.

Sustained release properties were displayed in Example 3, in that all of the samples gave the desired testosterone levels for about 7 h or more. However, each sample had one or two serum testosterone levels exceeding 1100 ng/dL.

### Example 4

### 100 mg Microencapsulated, 150 mg Micronized Testosterone Combined for 250 mg Dose

Four delivery systems of testosterone were prepared. Three samples contained microencapsulated micronized testosterone (100 mg). The three samples were microencapsulated with methylcellulose designed to release at either pH 5, 6 or 7. The remaining 150 mg of testosterone was micronized. The fourth sample was prepared with unencapsulated testosterone. The four samples were formulated into a lipid suspension as disclosed in Example 1 and given to four dogs. Serum levels of testosterone were measured as in Example 2.

TABLE 7
Serum Levels of Testosterone (ng/dL)

| Time (h) | Un-encapsulated | pH 5 Release | pH 6 Release | pH 7 Release |
|---|---|---|---|---|
| 0 | 105 | 5 | 12 | 15 |
| 1.5 | 488 | 166 | 314 | 254 |
| 3 | 626 | 179 | 333 | 290 |
| 6 | 496 | 426 | 487 | 271 |
| 9 | 125 | 438 | 599 | 348 |
| 12 | 79 | 576 | 377 | 344 |
| 15 | 34 | 351 | 266 | 195 |
| 18 | 14 | 86 | 90 | 173 |
| 21 | 10 | 55 | 75 | 190 |
| 24 | 4 | 30 | 112 | 117 |

The microencapsulated samples provided the desired levels of serum testosterone for from 9.0 to 13.5 hours.

TABLE 8
Sustained Release Times
Partial Microencapsulation

| Testosterone Form | Sustained Release Time (h) |
|---|---|
| Un-encapsulated | 4.5 |
| pH 5 Release | 9 |
| pH 6 Release | 13.5 |
| pH 7 Release | 10.5 |

In Example 4, the microencapsulated samples displayed sustained release properties, with the longest sustained release time in the sample with a pH 6 release coating.

### 100 mg Microencapsulated, 150 mg Micronized Testosterone Combined for 250 mg Dose in a Lipid Suspension

A delivery system of testosterone was prepared as described in Example 4. The sample contained micronized testosterone (100 mg) microencapsulated with methylcellulose designed to release at pH 6. The remaining 150 mg of testosterone was micronized. The sample was formulated into a lipid suspension as disclosed in Example 1.

The sample was administered to four hypogonadal males and serum cholesterol was monitored for each patient as given below:

TABLE 9
Serum Levels of Testosterone (ng/dL)

| Time (h) | Patient A | Patient B | Patient C | Patient D |
|---|---|---|---|---|
| 0 | 245 | 207 | 3 | 225 |
| 0.25 | 228 | 219 | 26 | 202 |
| 0.5 | 230 | 207 | 9 | 219 |
| 1 | 253 | 265 | 9 | 288 |
| 2 | 311 | 346 | 6 | 490 |
| 4 | 452 | 351 | 66 | 518 |
| 6 | 576 | 282 | 255 | 415 |
| 8 | 478 | 262 | 230 | 403 |
| 12 | 351 | 276 | 109 | 354 |
| 16 | 259 | 274 | 86 | 438 |
| 20 | 308 | 302 | 72 | 438 |
| 24 | 253 | 311 | 98 | 432 |

For patients A, B and D, the Example 5 delivery system provided testosterone levels in the blood in the desired range for greater than 20 hours. For patient C, the testosterone levels were below that desired. It is noted that for patient C, the initial testosterone level was significantly below that of the other patients.

### Example 6

### 100 mg Microencapsulated, 150 mg Micronized Testosterone Combined for 250 mg Dose, without Lipid

A delivery system of testosterone was prepared without the lipid suspension. The sample contained micronized testosterone (100 mg) microencapsulated with methylcellulose designed to release at pH 6. The remaining 150 mg of testosterone was micronized. The testosterone was delivered in a gel capsule. The delivery system was ingested simultaneously with a solid lipid suspension dosage that did not contain testosterone.

The sample was administered to the four hypogonadal males of Example 5, and serum testosterone was monitored for each patient as given below:

TABLE 10
Serum Levels of Testosterone (ng/dL)

| Time (h) | Patient A | Patient B | Patient C | Patient D |
|---|---|---|---|---|
| 0 | 219 | 158 | 3 | 245 |
| 0.25 | 530 | 150 | 26 | 256 |
| 0.5 | 1040 | 199 | 127 | 472 |
| 1 | 873 | 271 | 268 | 685 |
| 2 | 795 | 262 | 305 | 746 |
| 4 | 737 | 418 | 291 | 599 |
| 6 | 674 | 276 | 291 | 564 |
| 8 | 582 | 251 | 230 | 458 |
| 12 | 348 | 230 | 124 | 386 |
| 16 | 325 | 207 | 81 | 395 |
| 20 | 308 | 265 | 69 | 409 |
| 24 | 311 | 288 | 58 | 325 |

Whereas the testosterone without the lipid suspension gave a wider variance in blood serum levels of testosterone, it did deliver testosterone in the desired levels for periods in excess of 20 hours for two of the patients, A and D.

1. An orally administered testosterone delivery system with sustained release properties, wherein one dose of the system delivers an effective dose of testosterone as measured by total serum testosterone in the range of about 250 to 1100 ng/dL for greater than about 7 hours.

2. An orally administered testosterone delivery system with sustained release properties, wherein one dose of the system demonstrates improved sustained release properties over the same dose of micronized testosterone wherein the micronized testosterone is administered as dry particles alone or in a gelatin capsule.

3. An orally administered testosterone delivery system with sustained release properties, wherein one dose of the system demonstrates at least a 10% increase in sustained release properties when compared to the same dose of micronized testosterone, wherein the micronized testosterone is administered as dry particles alone or in a gelatin capsule.

4. The delivery system of claim 1, wherein at least part of the testosterone is microencapsulated.

5. The delivery system of claim 1, wherein all of the testosterone is microencapsulated.

6. The delivery system of claim 4, wherein the testosterone is suspended in a lipid.

7. The delivery system of claim 6, wherein the lipid is a solid at room temperature.

8. A method for preparing an oral testosterone delivery system with sustained release properties comprising the steps of:
microencapsulating testosterone particles;
melting at least one lipid;
mixing in a surfactant to said melted lipid;
dry-mixing dry particles comprising at least one filler and said microencapsulated testosterone;
mixing the dry particles with said melted lipid to form a suspension such that said dry particles are continuously coated by said lipid such that said suspension exhibits pseudoplastic and/or thixotropic properties, and pouring or molding said suspension into a dosage form, and wherein the dry particles optionally include non-encapsulated testosterone particles.

9. The method of claim 8 in which said testosterone particles are microencapsulated with a filming agent.

10. The method of claim 9 in which said microencapsulated testosterone particles are micronized.

11. The method of claim 9 in which said testosterone particles are microencapsulated with a rupturing agent.

12. The method of claim 11 in which said rupturing agent is sodium starch glycolate.

13. The method of claim 12 in which said lipid source forms 20% to 40% by weight of said suspension, and said dry particles form 60% to 80% by weight of said suspension.

14. The method of claim 13 in which said fillers have a size ranging from 10 to 500 microns in diameter and comprise whey.

15. The method of claim 8 in which said lipid is selected from the group consisting of a hard butter, petroleum Wax, vegetable fat or animal stearines.

16. The method of claim 8 in which said lipid suspension contains a rupturing agent.

17. The method of claim 16 in which said rupturing agent is sodium starch glycolate.

18. The method of claim 17 in which the dry particles include artificial flavorings.

19. An oral testosterone delivery system having sustained release properties comprising:
A. at least one lipid;
B. at least one surfactant; and
C. dry particles;
wherein, the dry particles contain testosterone and at least one filler;
wherein, the dry particles are continuously coated with the lipid and form a homogenous suspension with the lipid;
wherein the suspension exhibits pseudoplastic and/or thixotropic properties; and
wherein the suspension is formed or shaped into the appropriate solid dosage form by molding or pouring the suspension when in a liquid or semi-liquid state.

20. The testosterone delivery system of claim 19 in which at least part of the testosterone is microencapsulated.

21. The testosterone delivery system of claim 20 in which said microencapsulated testosterone contains a rupturing agent.

22. An oral testosterone delivery system having sustained release properties comprising:
A. at least one lipid;
B. at least one surfactant; and
C. dry particles
wherein the dry particles contain testosterone and at least one filler;
wherein the dry particles are continuously coated with the lipid and form a homogenous suspension with the lipid;
wherein the suspension exhibits pseudoplastic and/or thixotropic properties;
wherein the suspension is formed or shaped into the appropriate solid dosage form by molding or pouring the suspension when in a liquid or semi-liquid state;
wherein at least part of said testosterone particles is present in said suspension as a microencapsulated particle.

23. The pharmaceutical delivery system of claim 22 in which said microencapsulated testosterone contain therein a rupturing agent.

24. The testosterone delivery system of claim 23 in which said rupturing agent comprises sodium starch glycolate.

25. A method for preparing an oral testosterone delivery system having sustained release properties comprising:
melting at least one lipid;
mixing in at least one surfactant to said melted lipid;
mixing in dry particles to said melted lipid and surfactant, wherein said dry particles contain testosterone, and at least one filler;
wherein the dry particles are continuously coated with the lipid and for a homogenous suspension with the lipid;
wherein the suspension exhibits pseudoplastic and/or thixotropic properties; and
wherein the suspension is formed or shaped into the appropriate dose by molding or pouring the suspension when in a liquid or semi-liquid state.

26. The testosterone delivery system of claim 25, wherein the system includes additional drugs, medicaments or food supplements.

27. The delivery system of claim 6, wherein the lipid is a liquid.

28. An orally administered testosterone delivery system with sustained release properties, wherein one dose of the system delivers an effective dose of testosterone as measured by total serum testosterone in the range of about 250 to 1100 ng/dL for greater than about 7 hours, wherein the testosterone is delivered as a tablet, capsule, bolus, liquid, suspension or a mixture.

29. The delivery system of claim 28, wherein at least part of the testosterone is microencapsulated.
